(19) United States
(12) Patent Application Publication
(10) Pub. No.: US 2003/0118924 A1
(43) Pub. Date: Jun. 26, 2003

(54) Diarylethene derivatives and photochromic thin film prepared using the same

(75) Inventors: Eun Kyoung Kim, Yusung-ku (KR); Hyowon Lee, Wanjoo-kun (KR)

Correspondence Address: Finnegan, Henderson, Farabow,, Garrett & Dunner, L.L.P., 1300 I Street, N.W., Washington, DC, 20005-3315, US

(73) Assignee: KOREA RESEARCH INSTITUTE OF CHEMICAL TECHNOLOGY

(21) Appl. No.: 10/206,203

(22) Filed: Jul. 29, 2002

(30) Foreign Application Priority Data
Jul. 30, 2001 (KR) 2001-0045954

(51) Int. Cl. G11B 7/24; C07D 207/00; C07D 209/04; C07D 307/93; C07D 307/87; C07D 333/02; C07D 333/52

(52) U.S. Cl. 430/19; 430/945; 548/400; 548/469; 549/29; 549/49; 549/429; 549/462

(57) ABSTRACT

The present invention relates to a diarylethene derivative and a photochromic thin film using the same. More particularly, it relates to a diarylethene derivative and a photochromic thin film having excellent transparency prepared by depositing the same on the surface of a substrate, without crystallization or agglomeration to have effective photochromic property. This method also provides homogenous thin photochromic films with film thickness of a nano meter level and improved processability compared to conventional deposition processes thus, being suitable for coloring change with light repeatedly, optical information recording systems, and optical communication media.

### FIELD OF THE INVENTION

The present invention relates to a diarylethene derivative and a photochromic thin film using the same. More particularly, it relates to a diarylethene derivative and a photochromic thin film having excellent transparency prepared by depositing the same on the surface of a substrate, without crystallization or agglomeration, to have effective photochromic property. In addition, the present invention provides homogenous thin photochromic films with film thickness of a nano meter level and improved processability compared to conventional deposition processes thus, being suitable for reversible photochromic change, optical information recording, and optical communication media, etc.

### BACKGROUND OF THE INVENITON

Demand in photochromic lenses, high density photochromic recordings, high speed optical communications, and highly integrated circuits has been dramatically increased with development of technologies in shielding of sunlight, optical signal processing, optical transfer, optical filter and the like. The optical devices containing photochromic materials undergo a change in color and transparency with light irradiation and are capable of signal recording and/or reproducing with laser irradiation.

Conventional photochromic thin films have been prepared by dispersing a photochromic compound into a polymeric composition having a crosslinkable monomer, a polymer, a binder, and a curing initiator in a solvent, and than coating the composition on the substrate surface, followed by evaporation of the solvent or performing a crosslinking polymerization, by light curing or thermal curing. For example, Korean Patent Publication No. 2000-075550 discloses a method for preparing photochromic polyurethane thin film by a solvent coating and Korean Patent Publication No. 2000-016583 discloses a method for forming a recording layer by polymerizing a mixture of a polymerisable thermoplastic or curable binder containing a photochromic compound. However, when a photochromic compound is used for preparing a thin film with a polymer resin, it is difficult to obtain a homogeneous thin film due to insufficient compatibility of the photochromic compound with the polymer resin. Thus sufficient photochromic effect could not be observed since the concentration of photochromic compound in the polymer resin is low. On the other hand, when a large amount of a photochromic compound is used to enhance the efficiency, the obtained film is not clear and phase separation may occur with storing for a long period time because the photochromic molecules are separated out of the polymer resin or forms microcrystals. Therefore, it is unreliable and lack of storage stability for long term use. Further, when a photochromic compound is used with a crosslinkable monomer, the agglomeration among photochromic materials occur and photochromism becomes less effective due to poor polymerization. When photochromic compounds are crystallized by sublimation itself or recrystallization from a solvent, the particle size of the thin film is too fine to form large area of the film. Therefore, it has been limited to use photochromic thin films prepared by using compositions containing photochromic compounds as materials of recording medium, optical signal processing, and the like because the concentration of a photochromic compound in the thin film is too low to provide sufficient photochromic efficiency. Furthermore phase separation occurs during the thin film preparation, which leads to poor uniformity and errors in communications.

A deposition method has been introduced to resolve the problem associated with the conventional methods. However, it has been still unsatisfied to obtain the desired transparency of the thin film, because organic photochromic compounds aggregates unexpectedly and decomposition temperature thereof is usually lower than 200° C. In order to be free from these problems, Japanese Patent Publication No. 08-069083 discloses a method for preparing a thin film of diarylethene-based photochromic compound by separating a coloring material from the diarylethene-based photochromic compound using chromatography and vacuum vapor deposition on the substrate. However, it requires use of ultraviolet light which needs extra caution to handle, a separation process of a colored isomer from a colorless isomer. In addition an external light is required during the deposition of the colored isomer, thus being difficult for practical use.

### SUMMARY OF THE INVENTION

To provide solutions to the problems referred to above, inventors have studied to produce photochromic compounds having transparency capable of the deposition without employing chromatography process nor UV light irradiation. As a result, the inventors have developed diarylethene derivatives which can be maintained in a high concentration in the thin film without aggregation and does not decompose easily due to excellent thermal stability.

Accordingly, an object of the present invention is to provide a photochromic compound having excellent transparency, reversible photochromic characteristics, and control characteristics of optical signal with high efficiency. Another object of the present invention is to provide a photochromic thin film using the photochromic compound of the present invention.

### BRIEF DESCRIPTION OF THE DRAWING

The above and other objects and features of the present invention will become apparent from the following description of the invention, when taken in conjunction with the accompanying drawing, in which:

FIG. 1 represents absorption spectra of the photochromic thin film of the present invention before and after the irradiation of ultraviolet light.

### DETAILED DESCRIPTION OF THE INVENTION

The present invention provides a photochromic diarylethene derivative expressed by the following formula (1),

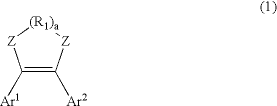

wherein R¹ is alkylene, or fluoro-substituted alkylene; a is an integer of 0˜3, Ar¹ and Ar² are independently a compound of formula (2) or a compound of formula (3); Z is fluoro-substituted or non-substituted methylene, carbonyl or CN when Z and Z is not linked by R′(a=0);

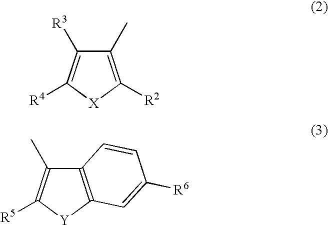

wherein R² and R⁵ are fluoro-substituted or non-substituted C₁-C₃ alkyl; R³ is the same as R² and R⁵, a hydrogen atom, or a fluoro atom; Both or one of R⁴ and R⁶ are independently acetyl, phenylisoxazole, hydroxymethylisoxazole, alkyleneoxyalkyl ester, aldehyde, carboxylic acid, —[CH═CH]₁—[C(═O)]ₘ[CH₂]ₙ—[CH═CH]ₒ—C(R⁷)ₚ(R⁸)_{q}, or —C≡C—R⁹, and one of R⁴ and R⁶ is the same as R³ when one of R⁴ and R⁶ are the group described above; R⁷, R⁸, and R⁹ are independently a hydrogen atom, C₁-C₂₂ alkyl or phenyl; l, n, and o are independently an integer of 0 to 10; m is 0 or 1; p and q are independently an integer of 0 to 3, and p+q=3; X and Y are independently an oxygen, nitrogen or sulfur atom.

The present invention is further to provide a photochromic thin film by depositing the photochromic compound of formula (1) of the present invention on the surface of a substrate.

The present invention is described in more detail as set forth hereunder. Photochromic diarylethene derivatives are represented in the following formula (1) and the photochromic diarylethene derivatives of formula (1) can be prepared by using known diarylethene compounds. For example, 1-(6′-benzylcarbonyl-2′-methylbenzo[b]thiophene-3′-yl)-2-(2″-methylbenzo[b]thophene-3″-yl)hexafluorocyclopentene of structure 2 is prepared by reacting phenylacetyl chloride, aluminum chloride (AlCl₃) and the compound of structure 1 in dry methylene chloride for 5 hrs at room temperature, followed by purification.

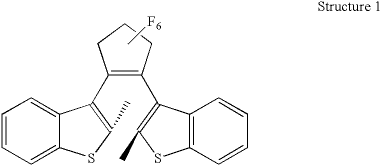

Particularly preferred diarylethene compounds of formula (1) include the following compounds of structure 2 to 20:

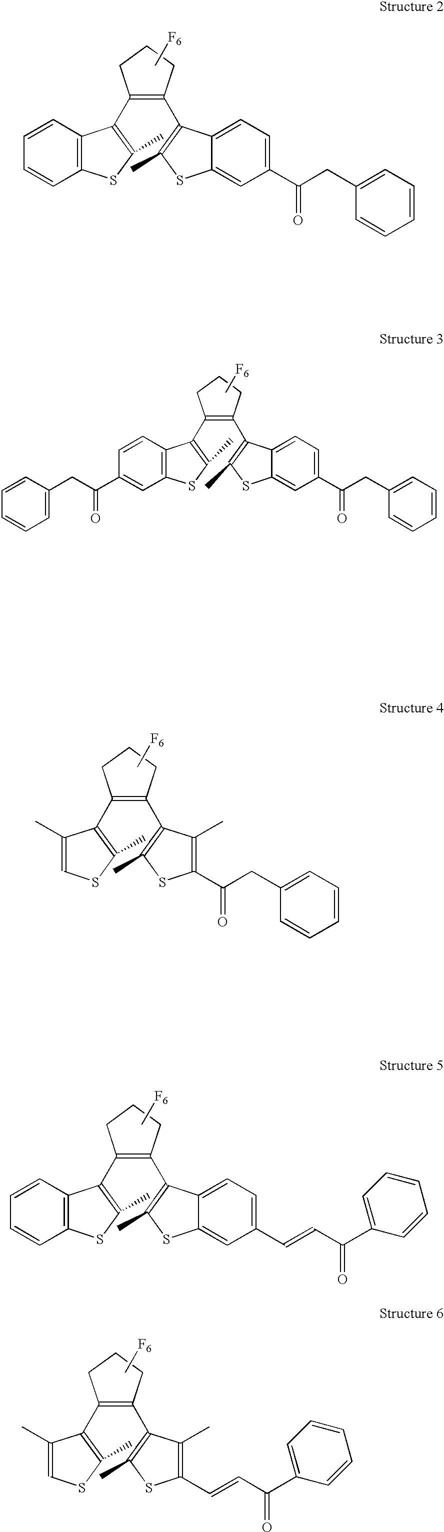

wherein R¹⁰ to R¹³ are the same as defined in formula 1 to formula 3.

In the diarylethene derivatives of formula (1), various substitutents of the compounds increase a random dispersion of molecules to prevent aggregation after the deposition. If the diarylethene derivatives do not have such substitutents, there is a drawback of becoming opaque with standing after the deposition due to molecular aggregation. Therefore, the present invention provides a transparent photochromic thin film without irradiating UV light to obtain high content of a colored isomer or running chromatography to separate a colored isomer from a colorless isomer. A thin film of photochromic layer can be prepared on plastic glass lenses, camera lenses and the like by a simple method according to the present invention.

Photochromic thin film of the present invention is prepared by depositing at least one diarylethene compound chosen from the diarylethene derivatives of formula (1). The diarylethene derivative is placed into a vacuum thermal evaporator and then deposited on the substrate preferably at a temperature range where decomposition does not occur. Examples of the substrate include glass, quartz, or glass or plastic plate coated with Al, AlCr, Au, or ITO. The thickness of the deposited thin film is 0.02 to 2 μm. The thermal evaporator is a general evaporator.

For example, the compound of structure 2 in a boat is deposited on the surface of quartz under the pressure at room temperature to provide a transparent thin film having 0.3 micron of a thickness.

During the deposition of the diarylethene compound on the substrate surface, additives such as a compound to form a thin film by the deposition, a compound to form a thin film by a polymerization with heat or light after the deposition, and a compound having photochromic property, used by one having ordinary skilled in the art, may be arbitrarily incorporated to improve functionality of the solution or physical properties of the thin film. At this time, the diarylethene compound and additives are mixed in the weight ratio of 100:0 to 0.5:99.5. Examples of the additive include dianhydrides such as pyromellitic dianhydride (PMDA), diamines such as 4,4-diaminodiphenyl ether (ODA), substituted or non-substituted paracyclophanes, substituted or non-substituted acetylenes and diacetylenes, naphthopyrans, phenanthropyrans, disperse Red, disperse Orange, fulgides, azobenzens and derivatives thereof, and spirobenzopyrans and derivatives thereof. These additives may be deposited with the diarylethene compound to form a thin film, or the diarylethene compound may be deposited before or after the additives to form a multi-layered thin film. Further, other additives, used by one having ordinary skilled in the art, such as a catalyst for polymerization, an initiator, an antioxidant, a coloring agent, may be arbitrarily incorporated.

These diarylethene compounds or compositions containing the diarylethene compound are applied on the various substrate including plastic lenses, switches, filters, glasses, quartz, and the like, to form the desired thin films and the methods and effects are as follows.

The photochromic thin film changes color to red, blue, violet, brown, or gray with UV light irradiation and returns to colorless with VIS light irradiation. Thus, it may be suitable for various applications such as glasses for shielding sunlight, vehicular and architectural windows, UV sensors, and the like by controlling UV/VIS light irradiation.

The photochromic thin film can be further applied to the plastic lenses by coating on the surface of the plastic lenses via the deposition. The plastic lenses may be coated by a general deposition. At this time, the photochromic thin film can be deposited to various lenses having different refractive indexes and shapes.

Moreover, the photochromic thin film can be applied to photochromic image films to record directly or through the mask by employing laser to the photochromic substrate. Examples of the substrate include silicon wafers, transparent plastic plates, glasses, quartz, and plates coated with a metal layer or Indium-Tin-Oxides (ITO) where the substrate is preferably deposited by coating. And then, the obtained photochromic thin film is exposed to UV or VIS light to record. For example, the photochromic thin film is exposed to He—Cd laser through a mask and the image pattern is transferred to the photochromic layer. Or, the photochromic thin film is exposed to UV light to have colored state and then exposed directly or indirectly to a visible laser to record an image by far field or near field recording methods. The recording mark formed can be interpreted using microscope, optical microscope, fluorescent microscope, confocal microscope, AFM, and infrared ray. The recording mark can also be erased completely using UV light or VIS light and the image does not degrade for more than 1 year.

The following Preparation Examples and Examples are intended to further illustrate the present invention without limiting its scope. Testing methods employed in measuring properties in the Examples are as follows.

[Method]

(1) Thickness: measured by using α-Step 200

(2) Photochromism: measured by using UV/VIS spectrophotometer

(3) Transparency: measured with UV/VIS spectrophotometer at 600 nm.

(4) Thermal property: measured by using Thermo Gravimetry Analysis and Differential Scanning Calorimetry

(5) Deposition: performed by using thermal evaporator system KVT-420 of Korea Vacuum Co.

### EXAMPLE 1

### Preparation of 1-(6′-benzylcarbonyl-2′-methylbenzo[b]thiophene-3′-yl)-2-(2″-methylbenzo[b]thophene-3″-yl)hexafluorocyclopentene(BCMBTFP, Structure 2)

To a compound of structure 1 (0.5 g) dissolved in 20 mL of dry methylene chloride (hereafter referred to as ‘MC’) was added phenylacetyl chloride (0.2 g). AlCl₃ (0.17 g, 1.28 mmol) was further added to the reaction mixture and reacted for 5 hours at room temperature. The reaction mixture was extracted with water and MC, and then an organic layer was washed with aqueous solutions of NaOH and NaCl, followed by drying over MgSO₄. The dried organic layer was evaporated under reduced the pressure to remove solvent, and the residue was purified by a flash column chromatography on silica gel (n-hexane/ethyl acetate=7/1 to 5/1) to obtain 1-(6′-benzylcarbonyl-2′-methylbenzo [b]thiophene-3′-yl)-2-(2″-methylbenzo[b]thophene-3″-yl)hexafluorocyclopentene (BCMBTFP, Structure 2) with 70% of yield.

¹H NMR (200 MHz, CDCl₃); δ2.19-2.66 (m, 6H), 4.33-4.25 (d, 2H, J=15.3), 7.16-7.31 (m, 7H), 7.58-8.03 (m, 4H), 8.26-8.37 (m, 1H), MS (m/z); 585 (M⁺, 24), 528 (65), 510 (20), 494 (100), 490 (8)

### EXAMPLE 2

### Preparation of 1-(6′-diphenyl acetyl-2′-methyl[b]thiophene-3′-yl)-2-(6″-diphenyl acetyl-2″-methyl[b]thophene-3″-yl)hexafluorocyclopentene (Structure 11)

To a compound of structure 1 (1 g) dissolved in 40 mL of dry MC was added diphenylacetyl chloride (1.45 g). AlCl₃ (0.85 g) was further added to the reaction mixture and reacted for 5 hours at room temperature. The reaction mixture was extracted with water and MC, and then an organic layer was washed with aqueous solutions of NaOH and NaCl, followed by drying over MgSO₄. The dried organic layer was evaporated under the reduced pressure to remove solvent and the residue was purified by a flash column chromatography on silica gel (n-hexane/ethyl acetate=8/1 to 5/1) to obtain the desired compound of structure 11(1.56 g, 1.82 mmol) with 85% of yield.

¹H NMR (200 MHz, CDCl₃); δ2.18 (s, 3H), 2.44 (s, 3H), 7.16-7.58 (m, 28H)

### EXAMPLE 3

### Preparation of 1-(6′-(4,4-dimethyl-3-oxo-1-pentene)-2′-methylbenzo[b] thiophene-3′-yl)-2-(2″-methylbenzo[b]thophene-3″-yl)hexafluorocyclopentene (Structure 8)

Pinacolone (0.15 g) was dissolved in 10 mL of dry THF and 0.9 mL of lithium diisopropylamide (2.0 M) was added thereto at a temperature of 0 to −10° C. After stirring the mixture for 1 hour, 1-(6′-formyl-2′-methyl benzo[b]thiophen-3′-yl)-2-(2″-methylbenzo[b]thophene-3″-yl)hexafluorocyclopentene (FMBTFP) (0.5 g, 1.0 mmol) dissolved in 10 mL of dry THF was added to thereto. The reaction mixture was reacted for 3 hours at room temperature and then extracted with water and ethyl acetate. An organic layer was washed with an aqueous solution of NaCl, followed by drying over MgSO₄. The dried organic layer was evaporated under the reduced pressure to remove solvent and the residue was purified by a flash column chromatography on silica gel (n-hexane/ethyl acetate=9/1) to obtain 1-(6′-(4,4-dimethyl-3-oxo-1-pentenyl)-2′-methylbenzo[b]thiophene-3′-yl)-2-(2&quot;-methylbenzo[b]thophene-3″-yl)hexafluorocyclopentene (Structure 8) with 72% of yield.

¹H NMR (200 MHz, CDCl₃); δ1.25 (s, 9H), 2.22 (s, 3H), 2.50 (s, 3H), 7.13-7.42 (m, 4H), 7.54-7.86 (m, 5H), MS (m/z); 578 (M⁺, 23), 537 (5), 521 (100), 501 (11), 477 (4)

### EXAMPLE 4

### Preparation of 1-(6′-benzoyl ethylene-2′-methylbenzo[b]thiophene-3′-yl)-2-(2″-methylbenzo[b]thophene-3″-yl)hexafluorocyclopentene (Structure 5)

Acetophenone (0.3 g) was dissolved in 20 mL of dry THF and 1.5 mL of lithium diisopropylamide (2.0 M) was added thereto at a temperature of 0 to −10° C. After stirring the mixture for 1 hour, 1-(6′-formyl-2′-methylbenzo[b]thiophen-3′-yl)-2-(2″-methylbenzo[b]thophene-3″-yl)hexafluorocyclopentene(FMBTFP, 1.0 g) dissolved in 20 mL of dry THF was added to thereto. The reaction mixture was reacted for 3 hours at room temperature and then extracted with water and ethyl acetate. An organic layer was washed with an aqueous solution of NaCl, followed by drying over MgSO₄. The dried organic layer was evaporated under the reduced pressure to remove solvent and the residue was purified by a flash column chromatography on silica gel (n-hexane/ethyl acetate=7/1) to obtain 1-(6′-benzoyl ethylene-2′-methylbenzo[b]thiophene-3′-yl)-2-(2″-methylbenzo [b]thophene-3&quot;-yl)hexafluorocyclopentene (Structure 5) with 66% of yield.

¹H NMR (200 MHz, CDCl₃); δ2.15 (s, 3H), 2.49 (s, 3H), 7.25-7.64 (m, 10H), 7.92-7.95 (m, 4H), MS (m/z); 598 (M⁺, 2), 583 (1), 120 (5), 105 (100), 77 (56)

### EXAMPLES 5-10

Photochromic compounds were prepared by performing the reaction under the conditions in Table 1 and the results were summarized.

TABLE 1

| | | | Solvent/temp/ | | Yield | ¹H NMR (300 MHz, |
|---|---|---|---|---|---|---|
| Ex. | Method | Reactant | hr | Product | (%) | CDCl₃) |
| 5 | Ex. 2 | MTF6 | MC/r.t./7 | Structure | 75 | 2.06, 2.10, 2.29, 2.31, |
| | | | | 4 | | 4.10, 6.55, 7.18-7.43 |
| 6 | Ex. 4 | FMTF6, | THF/0 to − | Structure | 70 | 2.25, 7.24, 7.29, 7.62, |
| | | acetophenone | 10° C., r.t./5 | 5 | | 7.67, 6.96-8.07 |
| 7 | Ex. 1 | FMTF6, | THF/0 to − | Structure | 57 | 1.97, 2.06, 2.29, 2.36, |
| | | phenylacetyl | 10° C., r.t./8 | 6 | | 7.05, 7.10, 7.63, 7.69, |
| | | chloride | | | | 7.56-8.03 |
| 8 | Ex. 3 | FMTF6, | THF/0 to − | Structure | 80 | 1.20, 1.93, 2.00, 2.25, |
| | | pinacolone | 5° C./6 | 9 | | 2.32, 2.36, 6.49, 6.55, |
| | | | | | | 7.56, 7.52 |
| 9 | Ex. 2 | MTF6, | MC/r.t./10 | Structure | 88 | 2.18, 2.44, 7.16-7.58 |
| | | diphenyl | | 11 | | |
| | | acetyl | | | | |
| | | chloride | | | | |
| 10 | Ex. 2 | Structure 1 | MC/r.t./12 | Structure | 62 | 2.52, 3.98, 6.96-7.37 |
| | | | | 7 | | |

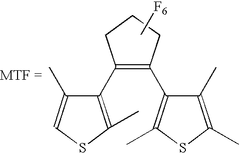

MC = Methylene chloride

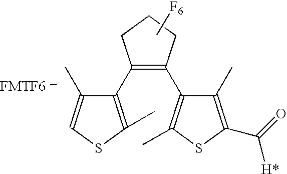

### EXAMPLE 11

### Preparation of Photochromic Thin Film

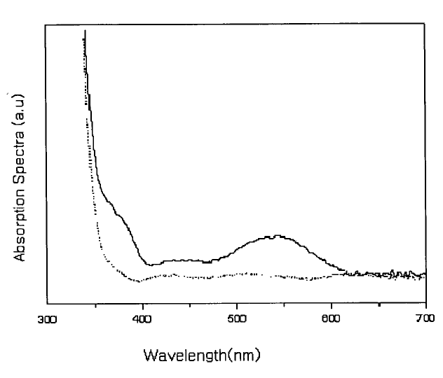

2.3 g of the photochromic compound prepared in Example 4 was placed in boat and transparent glass was used as a substrate. When an atmospheric pressure was reduced to 10-5 to 10-6, ampere meter was slowly increased from 1 and the photochromic compound was deposited on the surface of the glass with 20 □/s of a deposition speed to form a thin film with 550 nm thickness. When light was irradiated to the photochromic thin film obtained, color changed to red, while color was disappeared with VIS light irradiation. Such a photochromic property was represented in FIG. 1 which is absorption spectra of the photochromic thin film before and after the irradiation of ultraviolet light. According to FIG. 1, when UV light having a wavelength of shorter than 400 nm was irradiated, the plastic lens showed red color and a new absorption band was appeared. It is clearly understood that the thin film exhibits a photochromic property with light irradiation and it is reversible.

### EXAMPLES 12-20

The photochromic thin films were prepared by changing the compounds and deposition conditions in Example 11 as shown in the following Table 2.

TABLE 2

| | Diarylethene | | Deposition | Thickness | Contrast | Transparency after |
|---|---|---|---|---|---|---|
| Category | derivative (wt.%) | Substrate | speed (□/s) | (nm) | of color | 3 months at r.t. |
| Ex. 12 | Structure 2 (100) | Au-coated | 30 | 280 | 0.15 | Transparent |
| | | glass | | | | |
| Ex. 13 | Structure 2 (10), | Au-coated | 30 | 160 | 0.05 | Transparent |
| | Structure 13 (90) | glass | | | | |
| Ex. 14 | Structure 2 (5), Structure | Glass | 40 | 690 | 0.21 | Transparent |
| | 13 (55), SF (10), paracy | | | | | |
| | clophane (30) | | | | | |
| Ex. 15 | Structure 10 (50), | Glass | 30 | 340 | 0.17 | Transparent |
| | Structure 13 (50) | | | | | |
| Ex. 16 | Structure 12 (30), | Glass | 30 | 610 | 0.18 | Transparent |
| | Structure 10 (30), | | | | | |
| | PHDA (20), ODA (20) | | | | | |
| Ex. 17 | Structure 2 (5), DR-1(3), | Glass | 20 | 620 | 016 | Transparent |
| | Structure 13 (92) | | | | | |
| Ex. 18 | Structure 2 (10), | Glass | 20 | 580 | 0.13 | Transparent |
| | Structure 13 (90) | | | | | |
| Ex. 19 | Structure 2 (10), | Glass | 30 | 380 | 0.19 | Transparent |
| | Structure 12 (10), | | | | | |
| | Structure 18 (80) | | | | | |
| Ex. 20 | Structure 5 (10), | Glass | 25 | 630 | 0.21 | Transparent |
| | Structure 12 (90) | | | | | |
| Com. | Structure 1 | Glass | 30 | 350 | — | Cloudy |
| Ex. 1 | | | | | | |

SP: 6-hexyloxy phenyl-spirobenzopyran
DR-1: disperse Red 1

According to Table 2, the photochromic thin film using the diarylethene of the present invention exhibits excellent photochromic properties and transparency.

### EXAMPLE 21

### Preparation of Photochromic Image

The photochromic thin film prepared in Example 14 was exposed to the laser through a mask to form a photochromic image. Namely, UV light was irradiated on the photochromic thin film for 1 minute and the He—Ne layer was irradiated through the mask for 30 ms to form the photochromic image. The image was observed under an optical microscope and the resolution was higher than 100 nm. It was noted that the photochromic image provided red recording mark under UV light irradiation and the recording mark was disappeared under He—Ne laser. Further, it was proved that such a recording-fading property was repetitive and stable for more than 1 year.

As describe above, photochromic thin films having transparency using diarylethene derivatives of the present invention exhibit excellent photochromic property and homogeneous intensity even with a size of nanometer. In addition, processability of the present invention has been improved compared to the conventional deposition methods thus, being suitable for reversible photochromic color change, optical information recording systems, and optical communication media.

What is claimed is:

1. The present invention provides a photochromic diarylethene derivative expressed by the following formula (1), 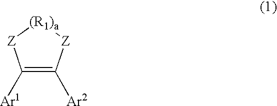
wherein R¹ is alkylene, or fluoro-substituted alkylene; a is an integer of 0˜3, Ar¹ and Ar² are independently a compound of formula (2) or a compound of formula (3); Z is fluoro-substituted or non-substituted methylene, carbonyl or CN when Z and Z is not linked by R′(a=0); 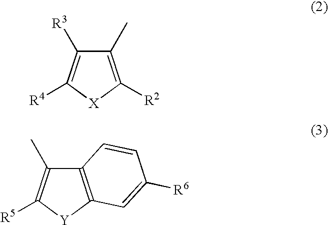
wherein R² and R⁵ are fluoro-substituted or non-substituted C₁-C₃ alkyl; R³ is the same as R² and R⁵, a hydrogen atom, or a fluoro atom; Both or one of R⁴ and R⁶ are independently acetyl, phenylisoxazole, hydroxymethylisoxazole, alkyleneoxyalkyl ester, aldehyde, carboxylic acid, —[CH═CH]ₗ—[C(═O)]ₘ[CH₂]ₙ—[CH═CH]ₒ—C(R⁷)ₚ(R⁸)_{q}, or —C≡C—R⁹, and one of R⁴ and R⁶ is the same as R³ when one of R⁴ and R⁶ are the group described above; R⁷, R⁸, and R⁹ are independently a hydrogen atom, C₁-C₂₂ alkyl or phenyl; l, n, and o are independently an integer of 0 to 10; m is 0 or 1; p and q are independently an integer of 0 to 3, and p+q=3; X and Y are independently an oxygen, nitrogen or sulfur atom.

2. A photochromic thin film prepared by depositing the compound of formula (1) on the substrate, 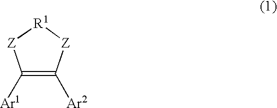
wherein R¹, Ar¹, Ar², a and Z are the same as defined in claim 1.

3. The photochromic thin film according to claim 2, wherein said photochromic thin film comprising 100 to 0.5 weight % of diarylethene derivative and 0 to 99.5 weight % of at least one additive selected from the group consisting of dianhydrides such as pyromellitic dianhydride (PMDA), diamines such as 4,4-diaminodiphenyl ether (ODA), substituted or non-substituted paracyclophane, substituted or non-substituted acetylenes and diacetylenes, naphthopyrans, phenanthropyrans, disperse Red, disperse Orange, fulgide, azobenzenes and derivatives thereof, and spirobenzopyrans and derivatives thereof.

4. The photochromic thin film according to claim 2, wherein said substrate is lenses, switches, filters, quartz or glasses.
